SEQUENCE LISTING (24 sequences)

SEQ ID NO: 1 - DNA, 29 bp, Artificial sequence
  Synthetic Primer
ggcgcgccaccatgggcgcgagaaactcc

SEQ ID NO: 2 - DNA, 42 bp, Artificial sequence
  Synthetic primer
gtacgtggctagcgtttaaacctatgccatttctccatcctc

SEQ ID NO: 3 - DNA, 29 bp, Artificial sequence
  Synthetic primer
gcaagcttggttcttgggttttctcgcaa

SEQ ID NO: 4 - DNA, 29 bp, Artificial sequence
  Synthetic primer
gcgatatcggtcctgtaggtacttctcta

SEQ ID NO: 5 - DNA, 27 bp, Artificial sequence
  Synthetic primer
ccaccatgaacgaaagggcagacgaag

SEQ ID NO: 6 - DNA, 18 bp, Artificial sequence
  Synthetic primer
ttgtctggtgtaggagac

SEQ ID NO: 7 - DNA, 46 bp, Artificial sequence
  Synthetic primer
gtcaagcgtctcctacaccagacaaatccatatccacaaggaccgg

SEQ ID NO: 8 - DNA, 30 bp, Artificial sequence
  Synthetic primer
atgccggggcccctaagtctcagccagtct

SEQ ID NO: 9 - DNA, 27 bp, Artificial sequence
  Synthetic primer
catatgggtcgctctgcggagaggctg

SEQ ID NO: 10 - DNA, 27 bp, Artificial sequence
  Synthetic primer
ccgcggacgcagacagtattaaaaaga

SEQ ID NO: 11 - DNA, 27 bp, Artificial sequence
  Synthetic primer
actagtggtcgctctgcggagaggctg

SEQ ID NO: 12 - DNA, 27 bp, Artificial sequence
  Synthetic primer
tacgtatgctagggattttcctgcctc

SEQ ID NO: 13 - DNA, 27 bp, Artificial sequence
  Synthetic primer
gaattcttttaaaagaagggggggaat

SEQ ID NO: 14 - DNA, 27 bp, Artificial sequence
  Synthetic primer
ctcgagaaaatcttttaattttgaatt

SEQ ID NO: 15 - DNA, 29 bp, Artificial sequence
  Synthetic primer
gcaagcttggttcttgggttttctcgcaa

SEQ ID NO: 16 - DNA, 29 bp, Artificial sequence
  Synthetic primer
gcgatatcggtcctgtaggtacttctcta

SEQ ID NO: 17 - DNA, 29 bp, Artificial sequence
  Synthetic primer
gcgcgcgcaatcaacctctggattacaaa

SEQ ID NO: 18 - DNA, 29 bp, Artificial sequence
  Synthetic primer
gcagatctcaggcggggaggcggcccaaa

SEQ ID NO: 19 - DNA, 28 bp, Artificial sequence
  Synthetic primer
gtttaaacgtaatcaattacggggtcat

SEQ ID NO: 20 - DNA, 31 bp, Artificial sequence
  Synthetic primer
gtcacttaagatctgacggttcactaaacca

SEQ ID NO: 21 - DNA, 29 bp, Artificial sequence
  Synthetic polycloning sequence
tcgagtttaaacgcgtcccgggtcgcgag

SEQ ID NO: 22 - DNA, 29 bp, Artificial sequence
  Synthetic polycloning sequence
cgcgctcgcgacccgggacgcgtttaaac

SEQ ID NO: 23 - DNA, 48 bp, Artificial sequence
  Synthetic polycloning sequence
ctagagcatgcgttaacgggcccggcgcgccgctagcggatccgagct

SEQ ID NO: 24 - DNA, 40 bp, Artificial sequence
  Synthetic polycloning sequence
cggatccgctagcggcgcgccgggcccgttaacgcatgct

(19) United States
(12) Patent Application Publication
(10) Pub. No.: US 2006/0134137 A1
(43) Pub. Date: Jun. 22, 2006

(54) Transduction vector based on modified HIV-2

(76) Inventors: James R. Gilbert, New Orleans, LA (US); Frank Park, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/015,131

(22) Filed: Dec. 17, 2004

(51) Int. Cl. C08B 11/193; A61K 39/12

(52) U.S. Cl. 424/204.1; 536/44

(57) ABSTRACT

A lentiviral vector system for gene transfer and gene therapy is disclosed. The system is based on human immunodeficiency virus type 2 (HIV-2), and uses a split-genome approach. The vector system can integrate into a host genome regardless of cell division. It may be used in terminally-differentiated cells. It has a high cloning capacity, between 8-11 kb. Minimal viral sequence is integrated into the host genome, reducing the likelihood that replication-competent viral particles will form following integration. HIV-2 is less pathogenic than HIV-1. By using different envelope proteins in the vector, the tropism may be targeted to particular cell types.

The development of this invention was funded in part by the Government under grant number 69620NIDDK awarded by the National Institutes of Health. The Government has certain rights in this invention.

This invention pertains to vectors for transducing the genomes of eukaryotic cells, including human and other mammalian cells, particularly to vectors based on the HIV-2 virus.

There is an unfilled need for improved vectors for transducing eukaryotic genomes, particularly mammalian genomes. Both viral and nonviral vectors have been used to transform and express exogenous genes into mammalian and other eukaryotic genomes.

Some prior vectors have been derived from retroviruses, such as the murine Moloney leukemia virus (MLV). Three characteristics of MLV have made it an attractive vector for gene therapy. First, MLV can stably integrate into target chromosomes, facilitating long-term expression of the integrated gene. Second, MLV vectors may be designed that do not transfer viral genes into target chromosomes. Furthermore, the viral particles themselves do not appear to cause cellular injury, nor to promote cytotoxic T cell-mediated cell death. Third, MLV has a relatively large cloning capacity (8-9 kb), facilitating the insertion of larger exogenous genes.

Although existing retroviral-derived transduction vectors have proven useful, they do have drawbacks. A major drawback of existing retroviral vectors is their general inability to enter the nucleus in the absence of cellular proliferation. Of course, the inability to enter the nucleus can be an advantage in circumstances where only transient expression is desired, rather than transduction of the genome. This property of retroviral vectors can also be an advantage where it is desired to selectively transform only proliferating cells. However, it is often desirable to non-transiently transform the genomes of cells that may or may not be dividing at the moment. Retroviral vectors suffer at least one additional drawback, namely that the transgene they carry may be silenced over time, known as position effect variegation. This “gene silencing” may be related to the site at which the retroviral vector integrates. It may be the case that the retroviral vector integrates into a transcriptionally active site during cell division. Later, when the cell returns to a quiescent state, the site of retroviral integration becomes inactive, perhaps because factors required to commence transcription are blocked from reaching the transgene. Whatever the mechanism, in many situations it is desirable for the transgene to remain active, and not to be silenced over time. (“Gene silencing” may also occur with lentiviral vectors.)

To try to overcome these drawbacks, some workers have designed replication-defective lentiviral vectors based on human immunodeficiency virus type 1 (HIV-1). See, e.g., L. Naldini et al., “In vivo gene delivery and stable transduction of nondividing cells by a lentiviral vector,” Science, vol. 272, pp. 263-267 (1996). Lentiviruses, which are a subfamily of the retroviruses, transduce a variety of cell types more efficiently than do their MLV counterparts, particularly cells that are not proliferating. Lentiviral vectors have the intrinsic ability to integrate into chromosomes, and to cause their transgene payload to be expressed without breaking down the nuclear membrane. These properties greatly facilitate in vivo gene transfer in quiescent cells.

For example, replication-incompetent lentiviral vectors have been reported to transduce non-dividing hepatocytes more efficiently than do MLV-based vectors. Nevertheless, if the hepatocytes are proliferating before they are exposed to the lentiviral transduction vectors, incorporation of the transgene into a chromosome is enhanced substantially. Because lentiviral vectors have the ability to transduce non-dividing cells more efficiently than MLV vectors, lentiviral vectors have been considered for possible uses in gene therapy.

K. Wang et al., “P75 interacts with the Nogo receptor as a co-receptor for Nogo, MAG and Mgp,” Nature, vol. 420, pp. 74-78 (2002) discloses a transduction vector based upon HIV-1.

One of the principal concerns with HIV-1 based vectors is the possibility that they might mobilize in individuals infected with wild-type HIV-1 virus, whether infected before or after the vector is administered. Some conditions that are potential targets for gene therapy, such as hemophilia, may even predispose patients to HIV-1. Testing the potential for vector mobilization is difficult in the absence of good animal models for HIV-1 infection. One approach to try to circumvent potential vector mobilization has been to design transduction vectors based on lentiviruses that do not normally infect humans, such as those whose natural hosts are felines, simians, or equines. However, human lentiviral vectors generally function more efficiently in human cells than do vectors based on lentiviruses from other species, as measured by titer levels.

Interestingly, even though human immunodeficiency virus 2 (HIV-2) is a human virus, its sequence has greater homology (˜75%) with the simian immunodeficiency virus (SIV) than with the similarly-named human virus HIV-1 (˜50%). Thus, despite similarity in nomenclature, and despite some similar properties, the HIV-1 and HIV-2 viruses are in fact only distantly related. The properties of one of these two viruses may not always be a reliable guide to the properties of the other.

Homologous elements of HIV-1 and HIV-2 often display nonreciprocal complementarity. In other words, a particular element from HIV-1 may sometimes function in HIV-2; however, it is not necessarily the case that the corresponding element from HIV-2 will function in HIV-1; and vice versa. Thus homology between HIV-1 and HIV-2 is not a reliable guide; what works with one virus will not necessarily work with the other.

Relatively little prior work has been conducted on HIV-2 generally, and in particular relatively little work has been done on engineered vectors derived from HIV-2. E. Poeschla et al., “Identification of a human immunodeficiency virus type 2 (HIV-2) encapsidation determinant and transduction of nondividing human cells by HIV-2-based lentivirus vector, “J. Virol., vol. 82, pp. 6527-6536 (1998) described an early-generation HIV-2 vector system. This system used a deletion between the major 5′ splice donor and the gag open reading frame, and was incorporated into a replication-defective, envelope-pseudotyped, three-plasmid HIV-2 lentivirus vector system that supplied HIV-2 Gag/Pol and accessory proteins in trans from an HIV-2 packaging plasmid. The vectors were reported to transduce marker genes into Human T and monocytoid cell lines, growth-arrested HeLa cells, and terminally differentiated human macrophages and NTN2 neurons. However, it appeared that hematopoietic progenitor cells were not stably transduced.

J. D'Costa et al., “Human immunodeficiency virus type 2 lentiviral vectors: packaging signal and splice donor in expression and encapsidation,” J. Gen. Virol., vol. 82, pp. 425-434 (2001) disclosed an HIV-2-based transduction vector system for transducing both dividing and non-dividing cells. Initial attempts yielded low titer. The system was then redesigned with a mutant splice donor site to produce vectors yielding a higher titer, and having higher vector RNA encapsidation.

It has been reported that HIV-2 itself may down-regulate HIV-1. See S. Arya et al., “Conditional regulatory elements of human immunodeficiency virus type 2 long terminal repeat,” J. Gen. Virol., vol. 75, pp. 2253-2260 (1994); S. Arya et al., “Human immunodeficiency virus (HIV) type 2-mediated inhibition of HIV type 1: a new approach to gene therapy of HIV-infection,” Proc. Natl. Acad. Sci. USA, vol. 93, pp. 4486-4491 (1996); and E. Kokkotou et al., “In vitro correlates of HIV-2 mediated HIV-1 protection,” Proc. Natl. Acad. Sci. USA, vol. 97, pp. 6797-6802 (2000).

L. Cheng et al., “Human immunodeficiency virus type 2 (HIV-2) vector-mediated in vivo gene transfer into adult rabbit retina,” Curr. Eye Res., vol. 24, pp. 196-201 (2002) reported the successful use of an early-generation HIV-2 transduction system in vivo in the rabbit eye by subretinal and intravitreal administration, but no assays were reported concerning any investigation into the potential for vector mobilization or recombination.

S. Arya et al., “Human immunodeficiency virus type 2 lentivirus vectors for gene transfer: expression and potential for helper virus-free packaging,” Hum. Gene Ther., vol. 9, pp. 1371-1380 (1998); J. D'Costa et al., “HIV-2 derived lentiviral vectors: gene transfer in Parkinson's and Fabry disease models in vitro,” J. Med. Virol., vol. 71, pp. 173-182 (2003); and S. Griffin et al., “The major human immunodeficiency virus type 2 (HIV-2) packaging signal is present on all HIV-2 RNA species: cotranslational RNA encapsidation and limitation of Gag protein confer specificity,” J. Virol., vol. 75, pp. 12058-12069 (2001) reported subsequent work with early-generation HIV-2 vector systems, but none reported any successful in vivo studies.

J. D'Costa et al. (2003) disclosed the development of monocistronic HIV-1 and HIV-2 vectors containing transgenes, vectors that were used to transform neuronal cells or Fabry fibroblasts in vitro.

S. Griffin et al. (2001) disclosed that the deletion of a 5′ leader from HIV-2 RNA reduced genomic encapsidation to about 5% of that for wild-type virus, with no defect in viral protein production, but severely limiting virus spread in Jurkat T cells.

T. Dull et al., “A third generation lentivirus vector with a conditional packaging system,” J. Virol., vol. 72, pp. 8463-8471 (1998) disclosed an HIV-1-based vector with several deletions made to attempt to improve biosafety.

Y. Bai et al., “Effective transduction and stable transgene expression in human blood cells by a third-generation lentiviral vector,” Gene Therapy, vol. 10, pp. 1446-1457 (2003) disclosed that difficulties in the genetic transduction of blood cells, including hematopoietic stem cells, had hampered the development of gene therapy applications for hematological disorders. The authors disclosed a self-inactivating vector based on HIV-1. High transduction efficiencies were reported with this system in human leukemia and myeloma cells in vitro.

J. Gilbert et al., “HIV-2 and SIV vector systems,” Somat. Cell Mol. Genetics, vol. 26, pp. 83-98 (2001); also published in G. Buchschacher (Ed.), Lentiviral Vector Systems for Gene Transfer (2003) reviewed HIV-2 and SIV-based transduction vectors, especially their use in transducing non-dividing cells. Unlike HIV-1, HIV-2 and SIV may be used in non-human primates. HIV-2 and SIV were also said to have the advantages of lower rates of transmission, and lower pathogenicity. See also K. Morris et al., “Transduction of cell lines and primary cells by FIV-packaged HIV vectors,” Mol. Ther., vol. 10, pp. 181-190 (in press, 2004).

Published United States patent application no. US2004/0147026 discloses a method for encapsidating transgene RNA using retroviral packaging and transfer vectors. An HIV-2 transfer vector, which includes the transgene, is introduced into a packaging cell that is also transfected with (or stably expresses) an HIV-2 derived packaging vector or a combination of packaging vectors. The packaging vector has mutations in packaging signal sequences that are both upstream and downstream of the 5′ splice donor site. The upstream mutation can be a functional deletion of a signal sequence located between the 5′ LTR and the 5′ splice donor site, while the downstream mutation can be a functional deletion of a signal sequence located between the 5′ splice donor site and an initiation codon of the gag gene on the HIV-2 genome. It can also be composed of a combination of two or more partial vectors. A transfer vector, which is introduced into the packaging cell line, has a mutation that renders its splice donor site non-functional. Transgene RNA expression and encapsidation from these cells is markedly increased, but with little or no levels of infectious viral RNA encapsidation. In particular embodiments, the packaging vector is an HIV-2(ROD) clone, such as pROD(SD36) or pROD(SD36/EM) plus pCM-ENV(ROD), the transfer vector is an HIV-2 clone, such as pSGT-5(SDM), and the packaging cell is a 293T cell.

We have discovered an advanced, efficient, “third-generation” transduction vector that is based on Human Immunodeficiency Virus type 2 (HIV-2). The benefits of the novel HIV-2 system include the following: (1) HIV-2 is known to be less pathogenic than its HIV-1 counterpart. (2) The vector is replication-defective, meaning that it is unlikely to become infectious, pathogenic, or to mobilize latent virus in the host. (3) The cell cycle arrest function is segregated from the nuclear localization function in the vpx gene product, which is analogous to the vpr gene from HIV-1. One of the accessory proteins of HIV-1, vpr, has dual functions. It induces cell cycle arrest in infected cells, which is an undesirable property for a vector. However, vpr also facilitates transport of the viral pre-integration complex across the nuclear membrane in the absence of cell division. This property is desirable in a vector, as it enhances the vector's ability to transfer a gene in a non-dividing cell. These two functions cannot be separated in HIV-1. By contrast, the two functions are associated with two separate accessory proteins in HIV-2. In HIV-2, vpr is responsible for causing cell cycle arrest, while vpx facilitates nuclear import. (4) Non-human primate models exist for HIV-2, but not HIV-1. The availability of such models not only facilitates studies of the efficacy of the vector in promoting genetic transductions in vivo, but it will also be useful in studying safety issues such as the potential for virus-vector recombination, and the potential for vector mobilization. (5) Optionally, the novel HIV-2 transfer vector may be cross-packaged with an HIV-1 packaging construct to minimize the likelihood of recombination, especially due to the limited sequence homology that exists between HIV-1 and HIV-2. (6) Because HIV-2 itself may down-regulate HIV-1, there is the possibility of dual protective effects if the novel vector is used for gene therapy of HIV-1 infection and AIDS. (7) Aside from HIV-2 and SIV, no other lentivirus-based vectors, including HIV-1-based vectors, can be tested effectively in a non-human primate model to assess the degree of vector mobilization that would be expected in humans. The safety profile of lentiviral vectors is, of course, an important consideration. Currently, there are no animal models that produce AIDS-like symptoms using HIV-1. However, simian immunodeficiency virus (SIV) causes an AIDS-like disease in non-human primates. SIV is highly homologous to HIV-2. Thus HIV-2-derived lentiviral vector systems may be used to assess the clinical safety profile of lentiviral vector systems generally, including lentiviral vectors derived from other species. (8) HIV-2 is less immunogenic than HIV-1, and is considerably less immunogenic than adenoviruses. Adenoviral vectors can themselves provoke an immune response in the host.

The novel vector will be useful in delivering therapeutic oligonucleotide constructs for long-term expression. As one example, the system may be used in the delivery of constructs for the treatment of diseases such as hemophilia or AIDS.

We have taken the HIV-2 provirus genome and divided it into separate parts. When these elements are simultaneously expressed in cell culture, “virions” are produced that are capable of transducing target cells, but whose construction renders them incapable of replication. The “virions” are capable of only a single round of gene transfer.

The novel system comprises three (or more) non-identical plasmids that, when co-expressed with a heterologous viral envelope, produce replication-defective viral particles. The system may be used to transfer exogenous DNA into a wide range of tissues and cell types, and to cause the exogenous DNA to be stably incorporated into the host genome. Note that, while many of the features of the novel system are derived from HIV-2, the viral envelope protein need not be from HIV-2, and may instead be any viral envelope protein that will bind to and be taken up by the target cells.

The novel system may be used for gene transfer generally, and for gene therapy in particular. Its biosafety features should offer advantages over other systems that are currently available. The novel system could also be used to supply “molecular therapies,” for example to AIDS patients: therapies such as ribozymes, anti-sense oligonucleotides, siRNA, TAR decoys, RRE decoys, or dominant-negative Revs.

Briefly, the so-called ψ sequence of HIV-2 is necessary for the viral RNA to be encapsidated. In the absence of ψ, the RNA will not be encapsidated. Two of the pieces (plasmids) into which we have split the viral genome lack the ψ sequence. These two segments are not incorporated into the virion, which is therefore replication-defective. The absence of either ψ-lacking segment alone would render a virion replication-defective. The absence of both segments further ensures that the virions will not replicate. Assays for replication competency have confirmed that the novel vectors are indeed not infectious. We have achieved high production level titers using vectors in accordance with this invention.

### BRIEF DESCRIPTION OF THE DRAWINGS

FIG. 1 depicts schematically the structure of a lentiviral virion, and of its RNA genome.

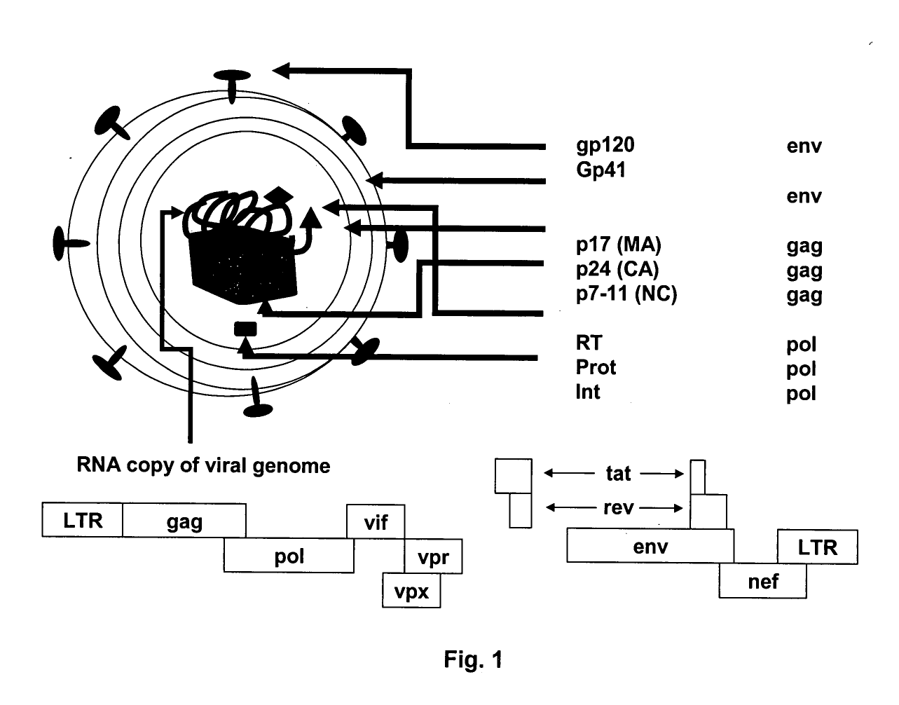

FIG. 2 depicts schematically, from top to bottom: a lentiviral genome, a packaging plasmid in accordance with the present invention, an envelope plasmid and a Rev-expressing plasmid in accordance with the present invention, and a transfer plasmid in accordance with the present invention.

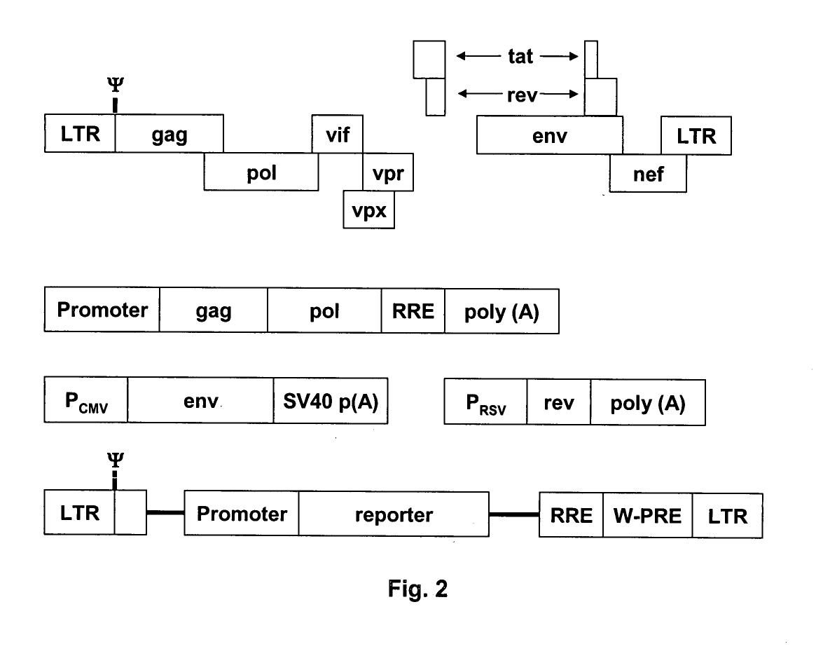

FIG. 3 depicts schematically the production of replication-defective lentiviral vectors with a split-genome packaging system in accordance with the present invention.

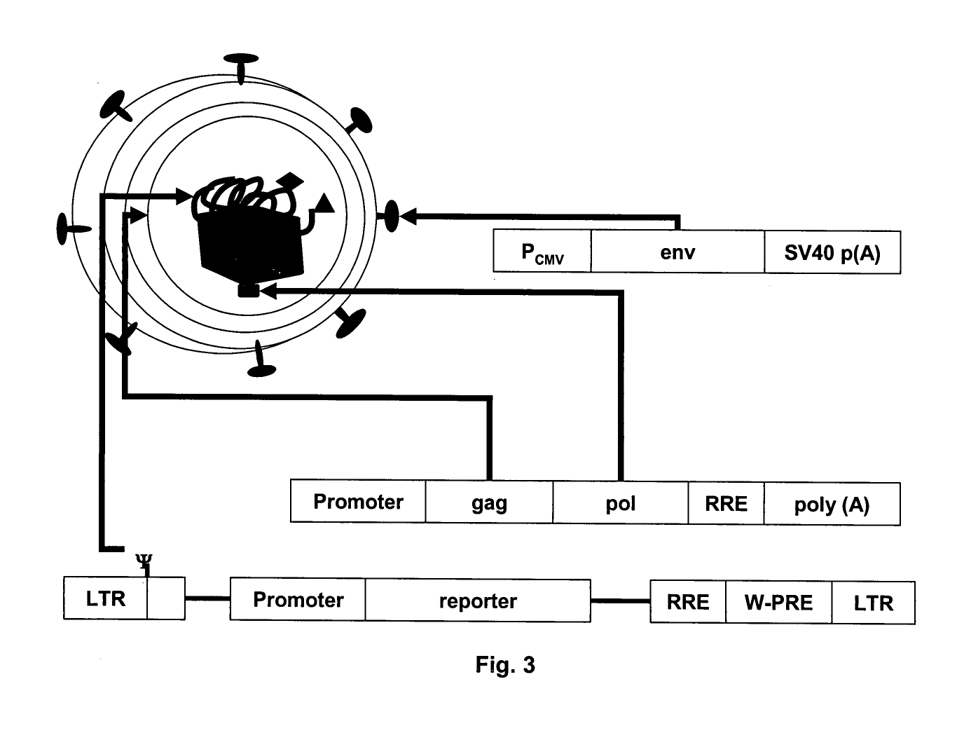

FIG. 4 depicts schematically, from top to bottom: a packaging plasmid and a Rev-expressing plasmid in accordance with the present invention, and a transfer plasmid in accordance with the present invention, where the transfer plasmid contains multiple polycloning sites.

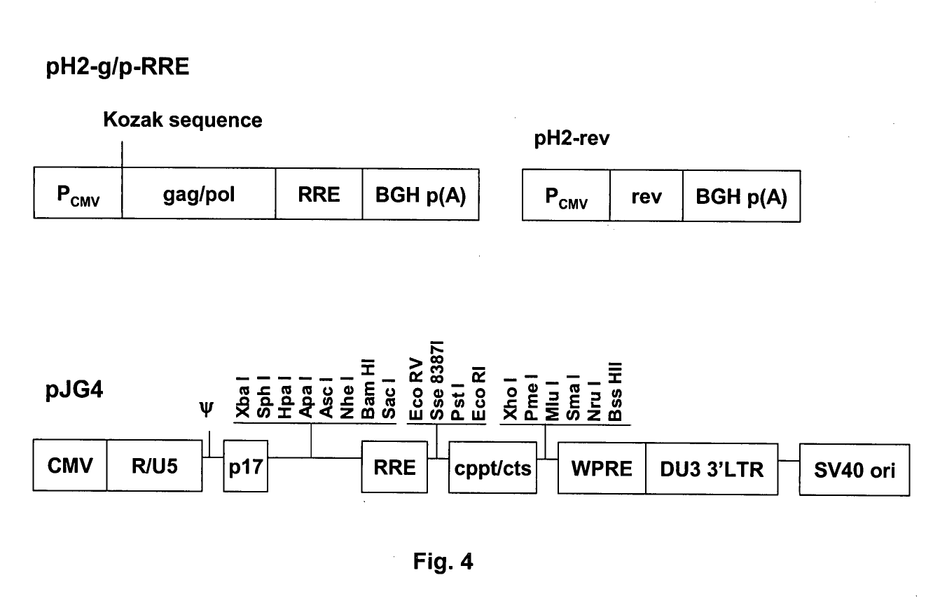

The gag and pol sequences in the first plasmid are preferably tightly defined, with no extraneous viral elements. (Frequently, when constructing a gag/pol expression plasmid, prior workers have taken advantage of existing restriction enzyme sites within the viral sequence. As a result, extraneous sequence flanking gag/pol has been incorporated into the plasmid, for example, elements of the viral long terminal repeat or fragments of viral accessory coding regions. In our preferred embodiment, the gag/pol coding region was cloned by PCR so that no extraneous viral elements were included. Furthermore, “first generation” HIV-2-based vectors generally had deleted only ψ and a portion of env. There was thus a greater risk of recombination into replication-competent virions with these earlier systems. See Poeschla et al. (1998).

In some “second generation” HIV-2-based vectors, in addition to the above “first generation” deletions, additional deletions were made in one or more of vif, vpr, vpx, and nef. See R. Zufferey et al., “Multiply attenuated lentiviral vector achieves efficient gene delivery in vivo,” Nat. Biotechnol., vol. 15, pp. 871-875 (1997).

In preferred embodiments of our “third generation” HIV-2-based vectors, in addition to deletions such as those used in “first generation” or “second generation” systems, one or more of the following changes (preferably all of the following changes) are made to the viral sequences: (a) using a hybrid promoter, formed by fusing a heterologous promoter to the 5′ viral long terminal repeat, thereby making the system independent of the viral tat protein; (b) incorporating a central polypurine tract (cppt) sequence into the transfer vector, which enhances the ability of the vector to infect non-dividing cells through a currently undetermined mechanism; (c) including the heterologous “woodchuck hepatitis virus post-transcriptional regulatory element” (W-PRE) in the transfer vector, enhancing transgene expression within transduced cells by stabilizing the polyadenylation of mRNA transcripts; (d) deleting the U3 region within the 3′ long terminal repeat, a form of “self-inactivating” deletion, in which the deletion silences expression of viral sequences, but not the transgene, in infected cells. By making these additional changes to the viral sequences, the probability that the construct will re-acquire replication competency is greatly reduced, nearly to zero.

When the Rev peptide is expressed, it binds to the viral RRE element, which is a short region possessing a high degree of secondary structure within the viral env gene. Binding of the Rev peptide to the viral RRE element stabilizes the mRNA, and facilitates nuclear export of unspliced transcripts. In a preferred embodiment, rev is placed on a separate plasmid from that containing gag and pol, further diminishing the potential for recombination. In the absence of the Rev expression product, the gag and pol RNA mRNA transcripts are degraded too rapidly for significant levels of translation.

In a preferred embodiment, we deleted most of the 5′ long terminal repeat, replacing it with the CMV early promoter.

Preferred embodiments may include one, several, or all of the following advantageous features: (1) The 3′ LTR is debilitated to substantially eliminate its weak promoter ability, making it unlikely that a full-length HIV-2 viral RNA will ever be transcribed after host genome integration. (2) One or more accessory genes (vif, vpr, vpx, and nef) are deleted, in whole or in part, thereby making a smaller fraction of the wild-type HIV-2 genome potentially available for recombination during vector production. Thus there is a reduced chance of producing replication-competent viruses. (3) The effects of removing the tat gene are temporarily alleviated by inserting a constitutive promoter at the 5′ end of the LTR. Potential modifications within the 5′ LTR can include deleting enhancer motifs, adding a core enhancer, and adding a TATA box for Tat to act as a transcriptional activator. By replacing this region within the LTR with a constitutive promoter, the need for tat may be eliminated. (4) The rev gene is placed on a separate plasmid from rev and pol, in a split-genome set-up that is unlikely to permit recombination either in vitro or in vivo. Tat is a viral protein that binds to the short TAR sequence within the viral long terminal repeat. When Tat binds, it enhances both transcriptional initiation and elongation, resulting in a 50-1,000 fold increase in expression of viral proteins. By inserting a strong, constitutive promoter upstream of the viral 5′ long terminal repeat, the need for Tat is eliminated. Deleting the Tat coding region may have value, because the protein has been shown to induce apoptotic cell death in several cell types. (5) Unique multiple cloning sites are inserted into the vector, making it easier to clone expression cassettes into the system. (6) In a preferred embodiment, the ROD strain of HIV-2 is used to generate the vector system, a strain that may help to produce high titers.
Some abbreviations used in the specification, figures, and claims include the following:
P_{CMV}=immediate early cytomegalovirus promoter (a strong, constitutive promoter)
gag/pol=lentiviral sequences expressing viral structural proteins and enzymes
BGH p(A)=bovine growth hormone polyadenylation signal
RRE=Rev Response Element
Rev=the viral Rev protein
p17=the first few hundred nucleotides of gag
cppt=central polypurine tract; enhances gene transfer in nondividing cells
W-PRE=woodchuck hepatitis virus post-transcriptional regulatory element; stabilizes RNA transcripts
eGFP=enhanced green fluorescent protein, a fluorescent reporter gene
eCFP=enhanced cyanoflourescent protein, another fluorescent reporter gene
ΔU3 3′LTR=deleted U3 in the 3′ long terminal repeat; causes inhibition of transcription from integrated double-stranded DNA, so viral proteins are not expressed.

The viral long terminal repeat (LTR) has three distinct regions, designated U3, R, and U5. U3 functions as the viral promoter. R contains a poly-adenylation signal. U5 contains enhancer elements. When a wild-type lentivirus integrates into the chromosome, it assumes a structure such as: 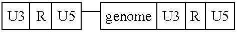
The 5′ U3 acts as promoter, driving viral gene expression.

However, when the virus is expressed as a full length genomic RNA, to be encapsidated within the budding virion, 5′ U3 and 3′ U5 sequences are lost, resulting in:

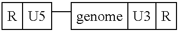

These “lost” sequences are replaced, via a template switching mechanism, when the virus infects subsequent cells.

### Plasmid Construction

### Packaging Constructs

### EXAMPLE 1

The HIV-2^{ROD} gag/pol coding region was PCR-amplified using 1 μg of Nco I-digested pSVR as template in a 100 μL reaction. The reaction mixture included 375 μM dNTPs, 10 μL Thermopol buffer (New England Biolabs), 1 μL of Vent DNA Polymerase, and 1 μg each of the oligonucleotides in the primer pair:

(SEQ ID NO 1)

```
                      Kozak
                    Sequence start
5′ GGC GCG CCA CCA TGG GCG CGA GAA ACT CC 3′
and
```

(SEQ ID NO 2)

stop
5′ GTA CGT GGCT AGC GTT TAA ACC TAT GCC ATT TCT CC
A TCC TC 3′

Twenty-five PCR amplification cycles were run: 97° C. for 30 seconds, 54° C. for 1 minute, and 72° C. for 4 minutes and 45 seconds. The 4,393 base pair PCR product was gel-purified, and was then cloned into the Eco RV site of pcDNA3 (Invitrogen) to generate the plasmid pcDNA3-g/p.

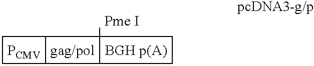

### EXAMPLE 2

The HIV-2^{ROD} rev-response element (RRE) was PCR-amplified using 1 μg of Nco I-digested pSVR as template in a 100 μL reaction. The reaction mixture included 375 μM dNTPs, 10 μL Thermopol buffer (New England Biolabs,) and 1 μL of Vent DNA Polymerase, and 1 μg each of the oligonucleotides in the primer pair:

(SEQ ID NO 3)

5′ GCA AGC TTG GTT CTT GGG TTT TCT CGC AA 3′
and

(SEQ ID NO 4)

5′ GCG ATA TCG GTC CTG TAG GTA CTT CTC TA 3′

Thirty PCR amplification cycles were run: 97° C. for 30 seconds, 54° C. for 1 minute, and 72° C. for 15 seconds. The 215 base pair PCR product was gel-purified, and was then cloned into the Pme I site of pcDNA3-g/p to generate pcDNA3-g/p-RRE.

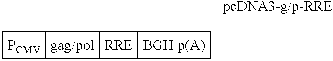

### EXAMPLE 3

The plasmid pcDNA-H2Rev was constructed sequentially. The first exon of rev was PCR-amplified using 1 μg of Nco I-digested pSVR as template in a 100 μL reaction. The reaction mixture included 375 μM dNTPs, 10 μL Thermopol buffer (New England Biolabs) and 1 μL of Vent DNA Polymerase, and 1 μg each of the oligonucleotides in the primer pair:

| | |
|---|---|
| Kozak | |
| Sequence start | |
| 5′ CCACCATGAACGAAAGGGCAGACGAAG 3′ | (SEQ ID NO 5) |
| and | |
| 5′ TTGTCTGGTGTAGGAGAC 3′ | (SEQ ID NO 6) |

Thirty PCR amplification cycles were run: 97° C. for 30 seconds, 54° C. for 1 minute, and 72° C. for 10 seconds. The 74 base pair PCR product was gel-purified, and was then cloned into the Eco RV site of pcDNA3 to generate pcDNA3-H2rev1.

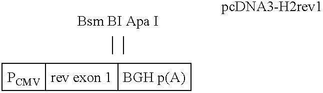

### EXAMPLE 4

The second exon of rev was PCR-amplified using 1 μg of Nco I-digested pSVR as template in a 100 μL reaction. The reaction mixture included 375 μM dNTPs, 10 μL Thermopol buffer (New England Biolabs), and 1 μL of Vent DNA Polymerase, and 1 μg each of the oligonucleotides in the primer pair:

(SEQ ID NO 7)

5′ GTCAAGCGTCTCCTACACCAGACAAATCCATATCCACAAGGACCGG
3′

(SEQ ID NO 8)

5′ ATGCCGGGGCCCCTAAGTCTCAGCCAGTCT 3′

The 232 base pair PCR product was sequentially digested with Bsm BI and Apa I. The digested product was cloned into the Bsm BI/Apa I backbone of pcDNAH2rev1 to create pcDNA-H2Rev.

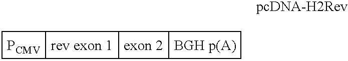

### Transfer Vectors

### EXAMPLE 5

The coding sequence spanning the 5′ long terminal repeat, leader region, and first 250 nucleotides of gag was PCR-amplified using 1 μg of Xba I-digested pSVR as template in a 100 μL reaction. The reaction mixture included 375 μM dNTPs, 10 μL Thermopol buffer (New England Biolabs) and 1 μL of Vent DNA Polymerase, and 1 μg each of the oligonucleotides in the primer pair:

| | |
|---|---|
| 5′ CATATGGGTCGCTCTGCGGAGAGGCTG 3′ | (SEQ ID NO 9) |
| and | |
| 5′ CCGCGGACGCAGACAGTATTAAAAAGA 3′ | (SEQ ID NO 10) |

The 807 base pair product was gel-purified, and was then cloned into the Stu I site of LITMUS28 (New England Biolabs) to generate LITMUS5′LTR.

### EXAMPLE 6

The R/U3 coding sequence of the 3′ long terminal repeat was PCR-amplified using 1 μg of Xba I-digested pSVR as template in a 100 μL reaction. The reaction mixture included 375 μM dNTPs, 10 μL Thermopol buffer (New England Biolabs) and 1 μg each of the oligonucleotides in the primer pair:

| | |
|---|---|
| 5′ ACTAGTGGTCGCTCTGCGGAGAGGCTG 3′ | (SEQ ID NO 11) |
| and | |
| 5′ TACGTATGCTAGGGATTTTCCTGCCTC 3′ | (SEQ ID NO 12) |

Twenty-five PCR amplification cycles were run: 97° C. for 30 seconds, 54° C. for 1 minute, and 72° C. for 30 seconds. The 298 base pair PCR product was gel-purified, and was then cloned into the Stu I site of LITMUS28 to generate LITMUS3′LTR.

LITMUS3′LTR was digested with Sna BI and Spe I, and the approximately 250 base pair sequence spanning the R/U3 region was gel-purified. This fragment was subsequently cloned into the Sna BI/Spe I backbone of LITMUS5′LTR to generate LITMUS 5′+3′LTR.

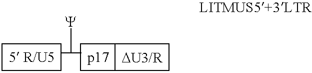

### EXAMPLE 7

The HIV-2 central polypurine tract (cppt) was PCR-amplified using 1 μg of Xba I digested pSVR as template in a 100 μL reaction. The reaction mixture included 375 μM dNTPs, 10 μL Thermopol buffer (New England Biolabs) and 1 μL of Vent DNA Polymerase, and 1 μg each of the oligonucleotides in the primer pair:

| | |
|---|---|
| 5′ GAATTCTTTTAAAAGAAGGGGGGGAAT 3′ | (SEQ ID NO 13) |
| and | |
| 5′ CTCGAGAAAATCTTTTAATTTTGAATT 3′ | (SEQ ID NO 14) |

Twenty-five PCR amplification cycles were run: 97° C. for 30 seconds, 54° C. for 1 minute, and 72° C. for 15 seconds. The 117 base pair PCR product was gel-purified, and was then cloned into the Stu I site of LITMUS28 to generate LITMUScppt.

LITMUScppt was digested with Eco RI and Xho I, and the 117 nucleotide sequence spanning the cppt was cloned into the Eco RI/Xho I backbone of LITMUS5′+3′LTR to create LITMUS-53-cppt.

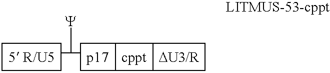

### EXAMPLE 8

The HIV-2^{ROD} Rev-response element (RRE) was PCR-amplified using 1 μg of Nco I digested pSVR as template in a 100 μL reaction. The reaction mixture included 375 μM dNTPs, 10 μL Thermopol buffer (New England Biolabs) and 1 μL of Vent DNA Polymerase, and 1 μg each of the oligonucleotides in the primer pair:

| | |
|---|---|
| 5′ GCAAGCTTGGTTCTTGGGTTTTCTCGCAA 3′ | (SEQ ID NO 15) |
| and | |
| 5′ GCGATATCGGTCCTGTAGGTACTTCTCTA 3′ | (SEQ ID NO 16) |

Twenty-five PCR amplification cycles were run: 97° C. for 30 seconds, 54° C. for 1 minute and 72° C. for 15 seconds. The 215 base pair PCR product was gel-purified, and was cloned into the Stu I site of LITMUS28 to generate LITMUSRRE.

### EXAMPLE 9

The woodchuck hepatitis virus post-transcriptional regulatory element (W-PRE) was PCR-amplified using 1 μg of Xba I digested as template in a 100 μL reaction. The reaction mixture included 375 μM dNTPs, 10 μL Thermopol buffer (New England Biolabs) and 1 μL of Vent DNA Polymerase, and 1 μg each of the oligonucleotides in the primer pair:

| | |
|---|---|
| 5′ GCGCGCGCAATCAACCTCTGGATTACAAA 3′ | (SEQ ID NO 17) |
| and | |
| 5′ GCAGATCTCAGGCGGGGAGGCGGCCCAAA 3′ | (SEQ ID NO 18) |

Twenty-five PCR amplification cycles were run: 97° C. for 30 seconds, 54° C. for 1 minute and 72° C. for 45 seconds. The 591 base pair PCR product was gel-purified, and was cloned into the Stu I site of LITMUS28 to generate LITMUS-WPRE.

Five μg of LITMUS-WPRE was digested with Bgl II/Bss HII, the 591 base pair fragment was gel-purified, and was cloned into the Bgl II/Bss HII backbone of LITMUS5′+3′-cppt to generate LITMUS-53-cppt-W⁺.

Forty μg of LITMUS-RRE was digested with Eco RI/Hind III, the 215 base pair fragment was gel-purified, and was cloned into the Eco RI/Hind III backbone of LITMUS-53-cppt-W⁺ to generate LITMUS-53-cppt-R⁺W⁺.

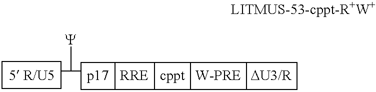

### EXAMPLE 10

The cytomegalovirus immediate-early promoter was PCR-amplified using 1 μg of Xba I-digested peGFP-N1 as template in a 100 μL reaction. The reaction mixture included 375 μM dNTPs, 10 μL Thermopol buffer (New England Biolabs) and 1 μL of Vent DNA Polymerase and 1 μg each of the oligonucleotides in the primer pair:

(SEQ ID NO 19)

5′ GTTTAAACGTAATCAATTACGGGGTCAT 3′
and

(SEQ ID NO 20)

5′ GTCACTTAAGATCTGACGGTTCACTAAACCA 3′

Twenty-five PCR amplification cycles were run: 97° C. for 30 seconds, 54° C. for 1 minute and 72° C. for 45 seconds. The 585 base pair PCR product was gel-purified, and was cloned into the Stu I site of LITMUS28 to generate LITMUS-CMV.

LITMUS-CMV was digested with Pme I/Afl II. The 585 base pair fragment was gel-purified, and was cloned into the Hpa I/Afl II backbone of LITMUS-53-cppt-R⁺W⁺ to generate pJG1CMV.

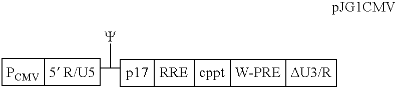

### EXAMPLE 11

To prepare reporter gene expression cassettes, the plasmids peCFP-N1 and peGFP-N1 (green fluorescent protein, Clontech) were each digested with Bam HI/Nhe I, and the 5′ overhangs of each plasmid were filled in with the Klenow fragment of DNA Polymerase I. The filled-in ends were subsequently re-ligated to produce the plasmids peCFP-N1ΔMCS and peGFP-N1ΔMCS, respectively. The plasmids peCFP-N1ΔMCS and peGFP-N1ΔMCS were each digested with Ase I, and the resulting 5′ overhangs were filled in the Klenow fragment of DNA Polymerase I. An Eco RI linker (New England Biolabs) was cloned into the filled Ase I site of peCFP-N1ΔMCS to produce peCFP-N1-Eco RI. An Xho I linker (New England Biolabs) was cloned into the filled Ase I site of peGFP-N1ΔMCS to produce peGFP-N-1-Xho.

The plasmid peGFP-N1-Xho was digested with Xho I/Hpa I and the 2,075 base pair fragment containing the CMV promoter followed by the eGFP reporter was gel-purified. This fragment was cloned into the Xho I/Bss HII backbone of pJG1′CMV (filled with Klenow) to produce pJG1CMV-GFP.

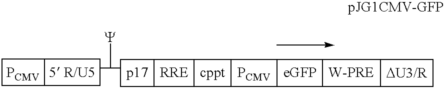

### EXAMPLE 12

To introduce multiple cloning sites into the transfer vector backbone, pJG1CMV was digested with Xho I/Bss HII. Equal amounts of the oligonucleotides:

| | |
|---|---|
| 5′ TCGAGTTTAAACGCGTCCCGGGTCGCGAG 3′ | (SEQ ID NO 21) |
| and | |
| 5′ CGCGCTCGCGACCCGGGACGCGTTTAAAC 3′ | (SEQ ID NO 22) |

were mixed, heated to 95° C., and subsequently cooled to 30° C. The annealed oligonucleotides were cloned into the Xho I/Bss HII backbone of pJG1CMV to produce pJG2CMV.

The plasmid pJG2CMV was digested with Xba I/Sac I. Equal amounts of the oligonucleotides:

(SEQ ID NO 23)

5′ CTAGAGCATGCGTTAACGGGCCCGGCGCGCCGCTAGCGGATCCGAGC
T 3′

(SEQ ID NO 24)

5′ CGGATCCGCTAGCGGCGCGCCGGGCCCGTTAACGCATGCT 3′

were mixed, heated to 95° C., and subsequently cooled to 30° C. The annealed oligonucleotides were cloned into the Xba I/Sac I backbone of pJG2CMV to produce pJG3CMV.

The plasmid peCFP-N1-Eco RI was digested with Eco RI/Ssp I, and the ˜2.3 kb fragment containing the CMV promoter, eGFP reporter, and SV40 polyadenylation signal was gel-purified. This fragment was cloned into the Eco RI/Eco RV backbone of pJG3CMV to produce pJG3CMV-CFP.

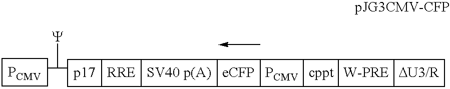

### Vector Production and Titering

### EXAMPLE 13

To produce vector supernatant, 293-T cells were seeded at a density of 2×10⁵ cells/mL in 10 cm dishes. The following day, cells were transferred to fresh media supplemented with 25 μM chloroquine. Thirty minutes later, 293-T cells were transiently transfected by standard calcium phosphate co-precipitation with the packaging plasmids pcDNA-g/p-RRE (7.5 μg/dish) and pcDNA-H2Rev (3.5 μg/dish), the envelope expression plasmid pMD.G (3.5 μg/dish), and the transfer vector pJG1CMV-GFP (10 μg/dish). Medium was replenished for the transfected cells after 24 hours, and the vector supernatants were harvested after an additional 24 hours. The supernatants were filtered through 0.45 μm syringe filters, and viral titers were determined on appropriate cell lines.

For titering purposes, HeLa and Huh7 cells were seeded at a density of 5×10⁴ cells/mL in 6-well plates. The following day, medium was replenished and was supplemented with 8 μg/mL polybrene. Serial dilutions of vector supernatants were added to individual wells in triplicate, and were incubated overnight. Forty-eight hours later the transduced cells were harvested, washed in PBS, and analyzed by fluorescent flow cytometry to determine vector titers, based on measured fluorescence from the reporter gene using FACS analysis.

Titer was calculated as (percentage of positive cells)×(dilution factor)×(total number of cells per well/100). For HeLa cells, the vector titer was found to be (1.62+/−0.01)×10⁶ infectious units/mL (i.u./mL) (n=3). For the Huh7 hepatoma cell line, the vector titer was found to be (2.38+/−0.66)×10⁶ i.u./mL (n=3).

The efficiency of gene transfer was also confirmed visually. Control HeLa cells were not treated with the vector. Experimental cells were HeLa cells transduced with the green fluorescent protein gene using the novel HIV-2 vector. Forty-eight hours after transduction, all cells were stained with DAPI, a nuclear dye, and were then examined by fluorescent microscopy. Individual nuclei of all cells fluoresced a neon blue due to the DAPI dye. None of the control cells fluoresced green. However, over 80% of the transformed cells fluoresced green, demonstrating that the transduction of the cells had been successful.

In future work, we will test an alternative embodiment in which a larger portion of gag is incorporated into the transfer vector, to determine whether this modification will improve packaging efficiency.

We will also confirm that, in the experiments described above, the reporter gene has been permanently incorporated into the genome, that expression is not merely transient, and that expression is not later silenced.

We will also directly compare gene transfer efficiencies for our novel HIV-2-based vector and for prior HIV-1-based vectors in the rat kidney. Vectors will be pseudotyped with viral envelope proteins from vesicular stomatitis virus G, Ross River virus, or Hantaan virus glycoproteins. Preliminary work using the envelope protein from vesicular stomatitis virus G have been promising.

### Initial Safety Assessment Studies

### EXAMPLE 14

Comparison of HIV-1-based vectors and HIV-2-based vectors; Safety and Gene Transfer Efficiency in rodents. This experiment is intended to directly demonstrate the reduced immunogenicity and pathogenicity of the novel HIV-2-based vectors as compared to HIV-1 vectors. Several viral envelopes are examined, as it is known that some envelopes, e.g., VSV-G, can themselves be cytotoxic. It is possible that liver and/or tissue damage resulting from VSV-G might be sufficiently high to mask more subtle distinctions between HIV-1 and HIV-2 vectors.

HIV-2 vectors and comparable HIV-1 vectors are prepared as previously described. These vectors incorporate a green fluorescent protein reporter gene, which is co-expressed with VSV-G, RRV, or Hantaan envelope expression plasmids in 293-T cells. Vector supernatants from these preparations are infused into the left kidney of adult male Sprague-Dawley rats (n=3). Blood from each animal is collected by retro-orbital bleed at 1, 3 and 7 days post-transfection. Serum ALT and AST are measured to assess any liver damage. Cytokine levels to assay any tissue damage are measured using a Bioplex Assay (BioRad). Experimental kidneys and control contra-lateral kidneys from each animal are harvested for tissue sectioning and measurements of gene transfer efficiency by fluorescent microscopy. Remaining tissues, including spleen, liver, heart, lung and brain are harvested and analyzed by real-time PCR to determine approximate vector spread.

### EXAMPLE 15

In Vitro Measurement of Vector Mobilization. Some studies have shown that vector mobilization may occur when cells that have previously been transduced with an HIV-1-based vector are subsequently infected with the HIV-1 provirus. To the inventors' knowledge, it has not previously been reported whether vector mobilization might occur in cells that have previously been transduced with an HIV-2-based vector that are subsequently infected with the HIV-1 provirus. While the HIV-2 provirus would be expected to mobilize an HIV-2 transfer vector, we do not expect the HIV-1 provirus to successfully mobilize an HIV-2 transfer vector. However, it is important to rule out this possibility. HIV-1 is relatively common in the western hemisphere; however, HIV-2 remains relatively rare in the western hemisphere.

A VSV-G pseudotyped HIV-2 vector, containing a GFP reporter gene, is prepared as described above. Primary peripheral blood lymphocytes (PBLs) are collected from human donors, and are activated with phytohemagglutanin A (PHA) and interleukin-2. Activated PBLs are transfected with the HIV-2 vector. After forty-eight hours, the PBLs are infected with a replication-competent strain of HIV-1 containing a CFP fusion reporter gene within the nef coding region. After an additional forty-eight hours, the culture supernatant is filtered through 0.45 micron filters to remove cellular debris. Primary human macrophage cells isolated from donors are subsequently infected with increasing doses of the filtered, virus-containing supernatant. Two days after infection, the macrophage cells are analyzed by flow cytometry to determine the abundance of GFP-, CFP-, or GFP/CFP-positive cells. Dual “staining” provides a measure of the frequency of vector mobilization in vitro.

### EXAMPLE 16

In Vitro Measurement of Vector Mobilization in Non-Human Primates: The next experiment addresses a similar question in vivo, in the most relevant non-human primate model currently available. HIV-2 vector-containing supernatant is prepared as described above. The vector-containing supernatant (10⁹, 10¹⁰, and 10¹¹ particles/animal) is infused into the livers of neonatal macaques (n=6). One week after vector administration, the macaques are infected with replication-competent SIV^{MAC} (n=3). Blood is harvested from each animal every 48 hours for two weeks, starting one day prior to infusion of the vector. After the initial two week period, blood is collected weekly for 6 months (or until euthanasia becomes necessary). Samples are examined for serum ALT and AST using standard assays, immunofluorescence, and flow cytometry to monitor CD4/CD8 counts. Viral load is assessed post-infection, both by p27 ELISA, and by determining the LD₅₀ on Jurkat cells. Vector mobilization is assessed by two methods. In the first method, flow cytometry is performed on PBLs isolated from each sample. In the second method, real time PCR is conducted on serum isolated from each sample using HIV-2-specific primers.

### Therapeutic Treatments

### EXAMPLE 17

Adenosine Deaminase Deficiency (ADA). Severe Combined Immunodeficiency resulting from ADA was one of the first disorders to be treated by gene therapy. Retroviral vectors were successfully used in clinical studies to deliver adenosine deaminase to hematopoietic stem cells. Gene expression has been detected up to 10 years after treatment, albeit at low, presumably sub-therapeutic levels. Lentiviral vectors should be more effective for this purpose, given their ability to transfer genes into both dividing and non-dividing cells. Hematopoietic stem cells (CD34(+)Thy-1(+)lin(−)) are isolated from ADA-SCID donors (n=3) using standard procedures. Approximately 10⁶ cells from each donor are transduced for 16 hours with an HIV-2-based vector as otherwise described above, containing a gene encoding human adenosine deaminase under the control of the phospho-glycero-kinase promoter, at a multiplicity of infection of 10. Transduced cells are collected by low speed centrifugation and washed repeatedly to eliminate residual virus. The washed, transduced cells are infused into autologous donors, and adenosine deaminase expression is monitored monthly for at least twelve months to confirm successful transformation and expression.

### EXAMPLE 18

Hemophilia. Hemophilia types A and B result from mutations in genes encoding blood clotting factor VIII and factor IX, respectively. It has been proposed that HIV-1-based vectors might be used to treat hemophilia. This approach is of questionable safety, however, due to the frequent co-incidence of HIV-1/AIDS in hemophiliacs. The novel HIV-2-based vectors are particularly well suited to treat hemophilia, however, to reduce potential hazards from vector mobilization. Using the novel HIV-2-based vector, liver cells are transduced with a gene encoding factor VIII or IX under the control of a cellular promoter, such as the phosphoglycerokinase promoter. Cells are transduced at a multiplicity of infection of about 5-10. Factor VIII or IX levels in the recipient's blood are assayed by ELISA bi-weekly basis for at least twelve months, to confirm the utility of the novel system for the beneficial treatment of hemophilia A and B.

### EXAMPLE 19

HIV-1/AIDS. Molecular strategies for treating HIV-1 infection have included proposals to use lentiviral vectors to deliver ribozymes, decoy RNA, anti-sense oligonucleotides, and siRNA. Few of these approaches would be compatible with the use of an HIV-1 vector. The therapeutic agent would inherently interfere with vector production. Due to the low degree of sequence homology between HIV-1 and HIV-2, however, HIV-2 vectors may be used to deliver therapeutic agents targeted against HIV-1. Hematopoietic stem cells (CD34(+)Thy-1(+)lin(−)) are isolated from suitable HIV-1 positive donors (n=3) using standard procedures. Approximately 10⁶ cells from each donor are transduced for 16 hours with an HIV-2-based vector as otherwise described above, expressing siRNA targeted against the HIV-1 pol coding region, under the control of the tRNA^{VAL} promoter, at a multiplicity of infection of 10. Transduced cells are collected by low speed centrifugation and are washed repeatedly to eliminate residual virus. Transduced cells will be infused into autologous donors. Blood is drawn from patients, and buffy coats are prepared weekly for at least six months. Expression of siRNA in PBLs and monocytes is confirmed by Northern analysis. CD4⁺/CD8⁺ counts is measured in all samples to assess effectiveness of therapy. HIV-1 expression is measured over time by p24 ELISA, and by Western analysis of protein extracted from buffy coats to assess the effectiveness of the therapy. The relative abundance of the transfer vector over time is assessed by real-time PCR.

### EXAMPLES 20 AND 21

Changing viral envelope proteins. Transducing cardiovascular cells. One advantage of using the lentiviral-based vector is the flexibility to incorporate envelope proteins from other viruses. Use of the VSV-G envelope protein may be limited in humans in vivo, due to its cellular toxicity and to its activation of the complement cascade, which can lead to partial inactivation of the vector. The VSV-G envelope has broad tropism for different cell types, a feature that can be either an advantage or a disadvantage, depending on the particular application for which the vector is being used. Not all cell types are transduced with the VSV-G envelope, however. For example, liver and endothelial cells are not transduced with the VSV-G envelope at high efficiency. Other viral envelope proteins may be used to tailor the tropism of the vector to particular target cells, for example lymphocytic choriomeningitis virus (LCMV) and Hantavirus (HTNV). Preliminary data from our laboratory (not shown), using an HIV-1-based viral vector system (rather than the HIV-2-based system disclosed here), suggest that the HTNV envelope protein is tropic for cardiovascular cells, particularly vascular smooth muscle and endothelial cells. We therefore expect that using the HTNV envelope protein in the HIV-2-based system of the present invention will target the vector towards cardiovascular cells.

The complete disclosures of all references cited in this specification are hereby incorporated by reference. In the event of an otherwise irreconcilable conflict, however, the present specification shall control.

We claim:

1. A construct for transducing both dividing and non-dividing mammalian cells; said construct comprising at least four different plasmids: a packaging plasmid, an envelope plasmid, a transfer plasmid, and a Rev-expressing plasmid; wherein:
(a) said packaging plasmid comprises a DNA oligonucleotide encoding HIV-2 gag, operably linked to a promoter that is functional in mammalian cells;
and said packaging plasmid comprises a DNA oligonucleotide encoding HIV-2 pol, operably linked to a promoter that is functional in mammalian cells; and said packaging plasmid does not comprise an HIV-2 ψ sequence; and said plasmid does not comprise a 3′ long terminal repeat; and said plasmid does not comprise a 5′ long terminal repeat;
(b) said envelope plasmid comprises a DNA oligonucleotide encoding a viral envelope protein, operably linked to a promoter that is functional in mammalian cells; wherein said envelope plasmid does not include any oligonucleotide that encodes any non-envelope HIV-1 or HIV-2, operably linked to a promoter that is functional in mammalian cells;
(c) said transfer plasmid comprises an HIV-2 5′ R element; and an HIV-2 5′ U5 long terminal repeat; and an HIV-2 3′ R element; and an HIV-2 ψ sequence; and either a DNA oligonucleotide sequence recognized by a restriction enzyme, adapted to accept a cloned transgene, or a transgene that encodes a protein whose expression is desired in mammalian cells that are transduced with said construct;
(d) said Rev-expressing plasmid comprises a DNA oligonucleotide encoding HIV-2 rev protein, operably linked to a promoter that is functional in mammalian cells; and
(e) said construct comprises oligonucleotides that encode zero elements or one element, but not more than one element, selected from the group of elements consisting of HIV-2 tat, HIV-2 vpr, HIV-2 vpx, HIV-2 vif, and HIV-2 net, operably linked to a promoter that is functional in mammalian cells.

2. A construct as recited in claim 1, wherein said construct comprises oligonucleotides that encode zero elements selected from the group consisting of HIV-2 tat, HIV-2 vpr, HIV-2 vpx, HIV-2 vif, and HIV-2 net, wherein the oligonucleotides are operably linked to a promoter that is functional in mammalian cells.

3. A construct as recited in claim 1, wherein said packaging plasmid additionally comprises one or more elements selected from the group consisting of a Kozak sequence, and a Rev response element.

4. A construct as recited in claim 1, wherein said transfer plasmid additionally comprises one or more elements selected from the group consisting of a p17 sequence, a Rev response element, cppt, wPRE, ΔU3, and SV40 ori.

5. A construct as recited in claim 1, wherein said construct is adapted for transducing both dividing and non-dividing human cells.

6. A construct as recited in claim 1, wherein said envelope plasmid comprises a DNA oligonucleotide encoding vesicular stomatitis virus G envelope protein.

7. A construct as recited in claim 1, wherein said envelope plasmid comprises a DNA oligonucleotide encoding hantavirus envelope protein.

8. A construct as recited in claim 7, wherein said construct is adapted for transducing both dividing and non-dividing human cardiovascular cells.
